(19) United States
(12) Patent Application Publication
(10) Pub. No.: US 2026/0210972 A1
(43) Pub. Date: Jul. 23, 2026

(54) CALIBRATION

(71) Applicant: Belgian Volition SRL, Isnes (BE)

(72) Inventors: Mark Edward Eccleston, Isnes (BE); Theresa K. Kelly, Isnes (BE)

(21) Appl. No.: 19/140,833

(22) PCT Filed: Dec. 19, 2023
(86) PCT No.: PCT/EP2023/086580

§ 371 Date: Jun. 18, 2025

Related U.S. Application Data
(60) Provisional application No. 63/433,682, filed on Dec. 19, 2022.

(51) Int. Cl. G01N 33/68; G01N 33/96

(52) U.S. Cl. CPC G01N 33/6875; G01N 33/96; G01N 2440/00; G01N 2474/20; G01N 2496/00

(57) ABSTRACT

The invention relates to methods for quantifying the amount of a nucleosome or a histone associated with a nucleosome in a biological sample wherein the method is calibrated using a nucleosome reference standard in a pre-calibration step.

### FIELD OF THE INVENTION

The present invention relates to the calibration of assays for cell free nucleosomes and related chromatin fragments.

### BACKGROUND OF THE INVENTION

Immunoassay methods for the estimation of nucleosomes are well known in the art, including the Cell Death ELISA (Enzyme Linked ImmunoSorbent Assay) marketed commercially by Roche Life Sciences (Holdenrieder et al, Int. J. Cancer (Pred. Oncol.): 95, 114-120, 2001) as well as assays developed by academic workers (Salgame et al, Nucleic Acids Research, 25(3), 680-681, 1997; van Nieuwenhuijze et al, Ann Rheum Dis; 62: 10-14, 2003).

Immunoassays are comparative analytical methods. In brief this means that the method does not directly establish the mass or concentration of an analyte present in a sample but provides a comparison to another sample or calibrant of known mass or concentration. As an analogy, a weighing balance is also a comparative analytical method in which the mass of a test item, on one arm of the balance, is compared to a calibrator weight on the other arm. The balance cannot directly determine the mass of the test item, but it can determine whether the mass is the same as the mass of the calibrator weight that it is compared to (for example a 1 kg weight). The determined mass of the test item will only be correct if the mass of the calibrator weight used is correct.

Immunoassays typically function by measuring the degree of antibody binding to an analyte and using this as a measure of the amount of analyte present. The degree of antibody binding is typically determined by labelling either the antibody or the antigen with a detectable tracer including for example, without limitation, a radioactive, colorimetric, chemiluminescent or fluorescent moiety which will give an assay output signal in radioactive counts, optical density (OD), relative light units (RLU) or fluorescence intensity respectively.

However, the immunoassay output signal is not a direct measurement of the mass or concentration of the analyte present in a test sample. In order to transform the assay output signal into a measure of concentration, for example in g/L or mol/L units, the signal is compared to that of a calibrant, or a series of calibrants, of known analyte concentration. The concentration of any test sample can then be determined by comparing the assay output signal observed for the test sample, to that observed for a calibrant that produces the same assay output signal in the immunoassay. In most cases a calibration curve, also often called a standard curve, is produced and the concentration of the sample is interpolated from the calibration curve. Thus, in order to provide a quantitative result, an immunoassay requires a calibrant to be used as a comparator and quantitative immunoassay kits usually include such a calibrant or a series of calibrants.

There is a continuing need to provide assays, including immunoassays, that are robust and provide a high level of precision. The present invention seeks to address this need.

### SUMMARY OF THE INVENTION

According to a first aspect of the invention, there is provided a method for quantifying the amount of a nucleosome or a histone associated with a nucleosome in a biological sample, the method comprising:
(i) providing a sample of nucleosomes at various concentrations to create a reference standard wherein the nucleosomes are assayed to generate a standard curve in a pre-calibration step;
(ii) adding a binding agent to a biological sample to perform an assay to measure the amount of nucleosome or histone associated with the nucleosome in the biological sample; and
(iii) quantifying the amount of nucleosome or histone associated with the nucleosome in the biological sample by measuring the amount of binding agent bound in the biological sample and comparing the amount to the reference standard.

According to a further aspect of the invention, there is provided a method for the measurement of the level of nucleosomes or histone associated with nucleosomes in a biological sample, the method comprising:
(i) obtaining a sample of a biological fluid;
(ii) contacting the sample with a binding agent to the nucleosomes;
(iii) measuring a parameter or degree of nucleosome binding of the binding agent to the nucleosome using an instrument adapted for conducting the assay;
(iv) comparing the parameter or degree of nucleosome binding obtained in step (iii) with standardisation data; and
(v) using the comparison in step (iv) to convert the parameter or degree of nucleosome binding measured in step (iii) into an amount or concentration of nucleosome present in the sample.

According to a further aspect of the invention, there is provided a kit for quantifying the amount of a nucleosome or a histone associated with a nucleosome in a biological sample wherein the kit comprises an automated immunoassay analyser, a nucleosome binder and electronically accessible standardisation data and does not comprise a nucleosome standard or calibrant.

According to a further aspect of the invention, there is provided a kit for measuring the level of nucleosomes or histone associated with nucleosomes in a biological sample, wherein the kit comprises an automated immunoassay analyser, a nucleosome binder and electronically accessible standardisation data and does not comprise a nucleosome standard or calibrant.

### BRIEF DESCRIPTION OF FIGURES

FIG. 1. 7 point standard curve run on freshly defrosted aliquots of recombinant H3.1 nucleosomes twice daily on 10 consecutive days.

### DETAILED DESCRIPTION

The present invention relates to a method employing a binding agent for the measurement of a nucleosome or a histone associated with a nucleosome in a biological sample from a subject, wherein said assay is calibrated using a nucleosome reference standard in a pre-calibration step. In particular embodiments the present invention relates to immunoassays.

Therefore, according to a first aspect of the invention, there is provided a method for quantifying the amount of a nucleosome or a histone associated with a nucleosome in a biological sample, the method comprising:
(i) providing a sample of nucleosomes at various concentrations to create a reference standard wherein the nucleosomes are assayed to generate a standard curve in a pre-calibration step;
(ii) adding a binding agent to a biological sample to perform an assay to measure the amount of nucleosome or histone associated with the nucleosome in the biological sample; and
(iii) quantifying the amount of nucleosome or histone associated with the nucleosome in the biological sample by measuring the amount of binding agent bound in the biological sample and comparing the amount to the reference standard.

Immunoassays are comparative measurement methods and require calibration using standard materials. A nucleosome immunoassay in its simplest form involves contacting an unknown sample with a binder of a nucleosome and measuring the degree of binding as an indicator of the level of nucleosomes present in the unknown sample. The degree of binding measured may be described as an assay response. This response must be compared to the response of a standard nucleosome preparation of known concentration to convert a nucleosome binding parameter to a nucleosome concentration.

Preparing and performing standard curves “wastes” time, materials and money for the users of commercially manufactured immunoassay methods. The person performing the assay must spend time preparing the standard materials. The standard materials are themselves expensive to manufacture. Moreover, performing a standard curve consumes assay reagents that cannot therefore be used for test samples. In an extreme example, 6 or 7 standard measurements may be made for comparative purposes to determine the level of nucleosomes present in a single test sample—thus multiplying the time, cost and effort required by a factor of 8.

The present invention obviates this waste by allowing the person performing a nucleosome immunoassay to omit the calibration or standardisation step of an assay altogether. Instead, the amount of binding agent bound in the biological sample is compared to the amount in a reference standard prepared in a pre-calibration step, i.e. a reference standard prepared in a separate method performed independently of the assay (optionally by a separate user).

The assay response of an immunoassay will vary with any changes in the parameters of the method employed, including for example small environmental fluctuations in temperature, humidity, air movement and incubation times as well as small fluctuations in reagent compositions and concentrations that occur in different reagent manufacturing batches. Performing a standard curve for each test of unknown samples ensures that any such small changes apply equally to standards and unknown samples thereby eliminating their effect.

Obviating nucleosome standard curves in the present invention may be achieved by automation of the method employed in a closed system in an instrument where all parameters such as temperature, humidity, air movement and incubation times are controlled. The remaining parameters relating to reagents are then accounted for by producing a standard curve for each manufacturing batch of reagents for use in the closed system. The standard curve may be produced on the same instrument as that used for testing unknown samples. The standard curve may then be stored for calibration purposes for any unknown samples analysed using that manufacturing batch of reagents. Alternatively the standard curve may be produced on another instrument which may be located anywhere in the world. For example, the standard curve may be produced remotely by the manufacturer of the reagents at the factory and communicated automatically, usually through an internet connection, to the instrument used for unknown sample analysis. In a preferred embodiment the manufacturing batch of any reagents used to analyse an unknown sample is recognised automatically by the instrument used, for example using a bar code system. The instrument then calls up the appropriate standard curve from a stored list of such curves or from a remote list provided by the manufacturer.

When running an immunoassay on ELISA plates, standards (or “calibrators”) are run at the same time, in the same plate, that the samples of interest are. This accounts for any variability in time and temperature that are inherent in manual colorimetric ELISAs. When immunoassays are run in a more automated format the overall precision of the timing and volume of the different steps is improved and there is less inherent variability than seen with manual assays. However, there is a continuing need to improve the efficiency and effectiveness of immunoassays. To this end we have developed an improved method that is robust and which is also capable of providing a high level of precision.

Removing the need for simultaneous calibration reduces the number of assays required for calibration improving capacity and throughput. For example, in a 96 well plate format plate typically two of the 8 well strips would be consumed for calibrating the plate reducing the number of available assays from 96 to 80, or 48 duplicates to 40. In addition performing calibration on a subset of units reduces the amounts of calibration material required.

In the present invention the standards and samples are run non-simultaneously.

For the avoidance of doubt “standards” and “calibrators” are used herein interchangeably.

According to another aspect of the invention, there is provided a method for quantifying the amount of a nucleosome or a histone associated with a nucleosome in a biological sample, the method comprising:
(i) adding a binding agent to a biological sample to perform an assay to measure the amount of nucleosome or histone associated with the nucleosome in the biological sample; and
(ii) quantifying the amount of nucleosome or histone associated with the nucleosome in the biological sample to a reference standard,
wherein the reference standard is generated in a pre-calibration step comprising providing a sample of nucleosomes at various concentrations to create a reference standard wherein the nucleosomes are assayed to generate a standard curve.

In one embodiment, the present invention employs a centralised instrument in which pre-calibration is conducted. For example, with some automated instruments, the calibration can valid for a certain period of time. Provided the lot number for the assays does not change, samples can be quantified with the same curve over this period of time. Preferably kit controls, e.g. two kit controls, are run each day to confirm they are in an acceptable range. The ensures acceptable precision and in this way the user can measure more samples for the same number of kits than using other methods.

In one embodiment of the invention the interval between calibration and conducting the assay with the sample is 10 days or more. This can be referred to as the “pre-calibration period”. In other embodiments this pre-calibration period is 12 days, 13 days, 14 days, 15 days, 21 days, 29 days or 36 days or more. In some embodiments, the pre-calibration period is between 10 and 90 days, such as between 14 and 60 days.

In one embodiment of the invention the interval between calibration and conducting the assay with the sample is about a week or more, such as about two weeks, about one month (30 days), about three months, about six months or more. In some embodiments, the pre-calibration period is between a week and six months, such as between two weeks and three months.

In another embodiment the present invention employs a point of care device in which a specific batch of units is manufactured and a subset of units is used to generate a standard curve at one location (e.g. the manufacturing site) which can be applied for quantification on the remaining batch by the end user at a remote site and time.

By “unit” we include a plate, cartridge, tube used in the assay.

In another embodiment pre-calibration by factory calibration is employed. Factory calibration as used herein refers to generating a standard curve during a calibration operation performed at a manufacturing facility or other factory stage before shipping to an end user.

In a particular embodiment, a batch code, identified through e.g. barcode in the assay, can be used to retrieve the appropriate standard curve.

In one embodiment, the method is performed on an automated analyser, such as an automated fluorescence analyser. The automated analyser may be capable of performing either one, or multiple types of assay. The automated analyser may be supplied with one or more test receptacles, e.g. a cuvette, a microtiter plate or a test cartridge, which are factor calibrated. The one or more test receptacles are supplied with the associated (i.e. lot- or batch-specific) calibration data. The associated calibration data may be provided on a storage device (for example a USB flash drive, hard-drive, memory card, DVD or CD-ROM) or may be downloaded from a remote location via a communication network (for example, the internet).

By way of example, one embodiment of the present invention may employ the LightDeck MINI System (hereafter referred to as “LightDeck MINI”) which is used in conjunction with LightDeck test cartridges. The LightDeck MINI and test cartridges are manufactured by MBio Diagnostics, Inc., based in Boulder, CO, USA.

As another example, one embodiment of the present invention may employ the IDS-iSYS which is a multiple-discipline automated analyser. Individual assays may be carried our in disposable cuvettes which are automatically loaded onto a carousel. Results are calculated in comparison to a calibration curve that is provided on a CD accompanying the test cartridge. This information is registered in the analyser's database when the CD is introduced on the controlling computer. The IDS-iSYS is supplied by Immunodiagnostic Systems Holdings Ltd, a PerkinElmer company, based in Tyne & Wear, UK.

### Calibrants

The present invention relates to the use of nucleosomes as standards for assay quantification. Nucleosomes that can be used the present invention include biologically derived nucleosomes and recombinant nucleosomes. In either case the nucleosomes will be assayed to generate a standard curve for assay quantification. This standard curve can be used to determine the amount or concentration of the nucleosome or histone associated with a nucleosome in the sample. References herein to “histone associated with a nucleosome” refer to histones and modifications thereof, as described herein (e.g. post-translational modifications, mutations, isoforms, variants and fragments of histones, such as clipped histones). Therefore, the standard curve can be used to determine the amount or concentration of one or more histone modifications, such as histone post-translational modifications, in the sample.

The nucleosome is the basic unit of chromatin structure and consists of a protein complex of eight highly conserved core histones (comprising of a pair of each of the histones H2A, H2B, H3 and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller, Methods Mol. Biol. (2007) 361: 25-62).

Recombinant nucleosomes are chemically synthesised nucleoproteins. Methods for the preparation of recombinant nucleosomes are known in the art and typically, involve the production of the individual recombinant core histones, chemically assembling the individual histones into histone octamers and binding the octamers to suitable lengths of DNA to form recombinant nucleosomes (Dyer et al, Methods in Enzymology, 375: 23-44, 2004). Typically, recombinant nucleosomes are pure single molecular complexes comprising a single histone isoform combination and are also uniform in terms of their post-translational histone modification composition. Thus, in one embodiment, the present invention relates to the use of recombinant/semi-synthetic nucleosomes carrying histone and/or DNA modification as standards for assay quantification in a pre-calibration step.

As used herein, a recombinant nucleosome (also called a designer nucleosome (dNuc)) is one that has been prepared by bringing together histones (including the core histones H2A, H2B, H3, and H4, and optionally linker histone HI), DNA, and optionally other factors to form the nucleosome. In other words, a recombinant nucleosome is one that is synthesized, not isolated from cells or chromatin. Each histone in the nucleosome may be independently fully synthetic, semi-synthetic (e.g., recombinantly produced and ligated to a synthetic peptide), or recombinantly produced. Each histone in the nucleosome may be a histone variant (e.g. H3.3, H2A.Bbd, H2A.Z.1, H2A.Z.2, H2A.X, mH2A1.1, mH2A1.2, mH2A2, or TH2B). The term recombinant nucleosome encompasses semi-synthetic nucleosomes and synthetic nucleosomes.

The recombinant nucleosome comprises a synthetic DNA sequence. The advantage of a synthetic DNA sequence is that it may be designed to include the desired properties. For example, the synthetic sequence may include a barcode sequence, a Widom nucleosome positioning sequence and/or a DNA sequence containing a disease specific single nucleotide polymorphism (SNP) (e.g. a KRAS). The length of the synthetic DNA sequence may be selected to provide nucleosomes with discrete DNA length e.g. 147 base pairs (bp), 167 bp or 187 bp of double stranded DNA. The synthetic DNA sequence can include a unique SNP or combination of SNPs to act as a barcode for identification. The synthetic DNA sequence can be blunt ended or selected to have a specific overhang whereby the overhang could represent a sequence specific to a physiological cleavage site enriched in a particular disease state e.g. cancer.

For the purposes of the present invention, a biologically derived nucleosome is a nucleosome that is not synthesised by chemically assembling recombinant histone and DNA components, but is a natural, cell derived nucleosome produced by a living cell. Therefore, the biologically derived nucleosome used as a calibrant, as described herein, is not a recombinant nucleosome. The terms “biologically derived nucleosome”, “cell derived nucleosome” and “natural nucleosome” are used interchangeably herein.

Biologically derived, or cell derived nucleosomes may be used in a raw form, but in embodiments of the invention the chromatin originating from cells is isolated, purified and fragmented (for example by sonication or nuclease digestion) to produce small chromatin fragments, including mononucleosomes and dinucleosomes, from larger cell derived chromatin fragments. In a further embodiment the biologically derived nucleosomes are cell free nucleosomes present in a body fluid, for example without limitation, lymph, cerebrospinal fluid, bronchoalveolar lavage fluid, blood, plasma or serum, wherein the cell free nucleosomes originate from (and were produced by) cells in the body. In another embodiment the biologically derived nucleosomes are cell free nucleosomes present in a cell culture supernatant, which cell free nucleosomes originate from (and were produced by) cells in culture. In one embodiment, the biologically derived nucleosomes are cell free nucleosomes present in extracellular trap (ET) material. Neutrophil extracellular trap (NET) or other ET material may be harvested from a number of sources including ex vivo culture of white blood cells stimulated to NETosis, or from body fluids such as blood, serum, plasma or other body fluids.

References to “nucleosome” may refer to “cell free nucleosome” when detected in body fluid samples. It will be appreciated that the term cell free nucleosome throughout this document is intended to include any cell free chromatin fragment that includes one or more nucleosomes. In addition, a cell free nucleosome may be a mononucleosome (analogous to a single “bead”), an oligonucleosome (analogous to a string of “beads”), part of a larger chromatin fragment or part of a NET or NET metabolite. Often the cell free nucleosomes present in a body fluid sample will be a mixture of some or all of these types.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, a histone isoform (also referred to herein as a histone variant), a protein bound to the nucleosome (i.e. a nucleosome-protein adduct), a DNA fragment associated with the nucleosome and/or a modified nucleotide associated with the nucleosome. For example, the component thereof may be histone (isoform) H3.1, histone H1 or DNA.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. In one embodiment, the measuring of nucleosomes per se comprises detecting a core histone protein, such as detecting histone H4. As described herein, histone proteins form structural units known as nucleosomes which are used to package DNA in eukaryotic cells.

Normal cell turnover in adult humans involves the creation by cell division of a huge number of cells daily and the death of a similar number, mainly by apoptosis. During the process of apoptosis chromatin is broken down into mononucleosomes and oligonucleosomes, some of which may be released into the circulation. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenrieder and Stieber, Critical Reviews in Clinical Laboratory Sciences; 46(1): 1-24, 2009).

A common application for immunoassays for cell free nucleosomes containing particular epigenetic structures or features, including histone PTMs or histone variants is the measurement of circulating cell free nucleosomes. Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in circulating nucleosome levels will be associated with elevations in circulating nucleosome subsets. Assays for these types of chromatin fragments are known in the art, including for example, those described in WO 2005/019826, WO 2013/030579, WO 2013/030578 and WO 2013/084002.

The test samples to be measured may be any sample comprising nucleosomes. In a preferred embodiment the test sample to be assayed by a comparative analytical method employing a calibrant or method of the invention is a human or animal body fluid sample including for example a blood, serum, plasma, cerebrospinal fluid, urine, faeces, sputum or saliva sample. Blood, serum or plasma samples are of particular interest. Therefore, in one embodiment, the biologically derived nucleosome preparation is used as a calibrant in a comparative analytical procedure which measures the level of histone post translational modifications in a blood, serum or plasma sample. Test samples may be prepared, for example where appropriate diluted or concentrated, and stored in the usual manner.

Immunoassay is a comparative analytical procedure in which the nucleosomes present in an unknown test sample may be measured using a binding agent that binds specifically to nucleosomes and the degree of binding is compared to the binding that occurs in a known calibrant or standard sample. Therefore, according to a further aspect of the invention there is provided an immunoassay method for the measurement of nucleosomes in a test sample, wherein said immunoassay is calibrated using a biologically derived nucleosome preparation assigned, or defined by, values expressed in absolute units of mass or concentration.

The biologically derived cell free nucleosome material for use as a calibrant may be produced from any biological material comprising chromatin. Common sources include chicken erythrocyte nucleosomes, circulating cell nucleosomes in blood, NETs and cell culture derived nucleosomes. Methods for the preparation of cell free nucleosomes from chicken erythrocytes and cultured cells are well known in the art. Chicken erythrocyte derived and cell culture derived nucleosomes are also available commercially, for example from Tebu-bio. Another potential source of nucleosomes includes heterophils, which are a type of granulocyte found in most avian species (e.g. chickens). Similar to neutrophils, heterophils can form heterophil extracellular traps (HETs). HETs released from chicken heterophils are structurally similar to NETs found in mammalian and fish neutrophils.

Many or most types of cells, particularly vertebrate cells, may be used as a source of chromatin material for the preparation of a biologically derived nucleosome calibrant. This is because most cells contain chromatin, and histone and nucleosome structures are highly conserved across species. This means that nucleosomes derived from any convenient animal source may be used as a nucleosome calibrant material for the absolute quantitation of nucleosomes in body fluid samples obtained from individuals of that species, or other species. For example, nucleosomes derived from any convenient animal source may be used as calibrant material for the absolute quantitation of nucleosomes in human body fluid samples. In one embodiment, the biologically derived nucleosomes are derived from calf thymus.

In one embodiment the nucleosomes may be provided diluted into the body fluid to be analysed. For example, animal derived nucleosomes may be provided in a matrix of human plasma.

In preferred embodiments, the biologically derived nucleosome material is produced from cells in cell culture (i.e. cultured cells). Methods for deriving cell free nucleosome material from cells in culture are well known (see for example Sadeh et al, Mol. Cell, 63(6): 1080-1088, 2016). In a typical method the chromatin material in the nucleus is isolated from the cells. This can be done using a nuclear isolation buffer or a commercially available nuclear isolation kit (for example the Merck, Nuclei PURE Prep kit). The chromatin material is then fragmented, typically by sonication to physically disrupt the chromatin, or by digestion using a nuclease (usually a micrococcal nuclease [MNase]). The chromatin fragments include mononucleosomes and/or oligonucleosomes. Optionally the chromatin may be cross-linked prior to preparation of fragmented chromatin by treating the cells with formaldehyde prior to isolation of the nuclear material. Cross-linking stabilises the fragmented chromatin and thus provides a more stable calibrant material. Alternatively, pre-prepared cross-linked fragmented chromatin materials produced from a variety of different cell types are available commercially including from Hela cervical cancer cells, HepG2 liver cancer cells, K562 leukaemia cells and 3T3 embryonic fibroblast cells. Any tissue or cultured cells may be used as a source of fragmented chromatin. Herein, we have used a chromatin fragment preparation produced from Hela cells.

Therefore, in one embodiment, the biologically derived nucleosomes are obtained from nuclease digests of cells grown in culture, said cells being immortalised human or animal cells or primary human or animal cells with native DNA, DNA modifications, histone variants and/or post translational modifications.

As a biologically derived nucleosome preparation contains a multiplicity of nucleosome types that comprise a wide variety of epigenetic features and combinations thereof, it will be understood that nucleosomes containing any particular epigenetic feature or features will form a subgroup of nucleosomes within the overall nucleosome composition of the biologically derived preparation.

It will be understood that more than one epigenetic feature of cell free nucleosomes may be detected by immunoassay or other comparative analytical methods. Multiple biomarkers are often used in medicine, for example to determine the disease status of a subject. One advantage of a biologically derived calibrant is that it comprises a mixture of a large number of different nucleosome types so a single biologically derived calibrant may be used as a combined calibrant for a large number of different nucleosome assays either separately or in a multiplex format. Therefore, in one embodiment, the biologically derived nucleosome preparation contains a combination of multiple different nucleosomes, and optionally other chromatin fragments, comprising multiple epigenetic features as a combined calibrant. In one embodiment the calibrant is a so-called universal, or near-universal, chromatin fragment calibrant that is suitable for use in all or most nucleosome or chromatin fragment assays. The epigenetic features useful in a calibrant may be of the same type (e.g. multiple PTM types, multiple histone isoforms, multiple nucleotides or multiple protein adducts) or different types (e.g. a PTM in combination with a histone isoform).

The structure of a nucleosome may vary by the inclusion of alternative histone isoforms or variants which are different gene or splice products and have different amino acid sequences. In one embodiment, the epigenetic feature of the nucleosome, such as of one or more of the multiplicity of nucleosome types, comprises a histone variant or isoform. Many histone isoforms are known in the art. Histone isoforms can be classed into a number of families which are subdivided into individual types. The sequences of a large number of histone isoforms are known and publicly available for example in the National Human Genome Research Institute NHGRI Histone Database (Marino-Ramirez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, isoforms of histone H2 include H2A1, H2A2, mH2A1, mH2A2, H2AX and H2AZ. In another example, histone isoforms of H3 include H3.1, H3.2 and H3t. In one embodiment, the histone isoform is H3.1.

Another way the structure of nucleosomes may vary is by mutation. Therefore, in one embodiment, the epigenetic feature is a mutated histone. In a further embodiment, the mutation is in histone 3 (H3). In a yet further embodiment, the mutation in H3 is when lysine 27 is replaced by a methionine (H3K27M).

The structure of nucleosomes can vary by post translational modification (PTM) of histone proteins. PTM of histone proteins typically occurs on the tails of the core histones and common modifications include acetylation, methylation or ubiquitination of lysine residues as well as citrullination or methylation of arginine residues and phosphorylation of serine residues and many others. It will be understood that a histone PTM may occur on different isoforms (variants) of the histone. For example, the lysine residues that occur on the tail of histone H3 isoforms H3.1, H3.2 and H3.3 may be modified by acetylation or methylation. Many histone modifications are known in the art and the number is increasing as new modifications are identified (Zhao and Garcia, Cold Spring Harb. Perspect. Biol. 7(9): a025064, 2015). Therefore, in one embodiment, the epigenetic feature of the cell free nucleosome may be a histone post translational modification (PTM). The histone PTM may be present on a core nucleosome histone (e.g. H2A, H2B, H3 or H4), or a linker histone (e.g. H1 or H5). Examples of PTMs are described in WO 2005/019826 and WO 2017/068359.

In one embodiment, the nucleosome preparation may contain particular histone PTMs including acetylation, methylation (which may be mono-, di- or tri-methylation), phosphorylation, ribosylation, citrullination, ubiquitination, hydroxylation, glycosylation, nitrosylation, glutamination and/or isomerisation. This may then be used to determine the level of histone post translational modification in a sample (e.g. a blood, serum or plasma sample from a patient). In one embodiment, the histone PTM is methylation of a lysine residue. In a further embodiment, the methylation is of a histone 3 lysine residue. In a yet further embodiment, the histone PTM is selected from H3K27Me3, H3KMe2, H3K4Me2 or H3K36Me3. In one embodiment, the histone PTM is acetylation of a lysine residue. In a further embodiment, the acetylation is of a histone 3 lysine residue. In a yet further embodiment, the histone PTM is selected from H3K9Ac, H3K14Ac, or H3K27Ac. In another embodiment, the histone PTM is H4PanAc. In one embodiment, the histone PTM is phosphorylation of a serine residue. In a further embodiment, the phosphorylation is of an isoform X of histone 2A (H2AX) serine residue or phosphorylation of a histone 3 serine residue. In a yet further embodiment, the histone PTM is selected from pH2AX or H3S10Ph. In one embodiment, the histone PTM is selected from citrullination or ribosylation. In a further embodiment, the histone PTM is citrullinated H3 (H3cit) or citrullinated H4 (H4cit). In a further embodiment, the histone PTM is citrullination of a histone 3 arginine residue. In a yet further embodiment, the histone PTM is H3R8Cit. In one embodiment, the histone PTM is selected from the group consisting of: H3K27Me3, H3KMe2, H3K4Me2, H3K36Me3, H3K9Ac, H3K14Ac, H3K27Ac, H4PanAc, pH2AX, H3S10Ph and H3R8Cit.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes and without limitation, anti-pan-acetylation antibodies (e.g. a Pan-acetyl H4 antibody [H4panAc]), anti-citrullination antibodies or anti-ubiquitin antibodies.

In one embodiment, the epigenetic feature is a DNA modification, thus the epigenetic feature of the nucleosome calibrant with a defined mass or concentration comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues, or 5-hydroxymethylcytosine residues or other nucleotides or modified nucleotides, than other nucleosomes. In one embodiment, the epigenetic feature is a DNA modification selected from 5-methylcytosine or 5-hydroxymethylcytosine. Thus, in some embodiments, the defined calibrated DNA modification is 5-methylcytosine or 5-hydroxymethylcytosine.

A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art. Therefore, in one embodiment, the epigenetic feature comprises one or more protein-nucleosome adducts or complexes. In a further embodiment, the epigenetic feature of the calibrant with a defined mass or concentration is one or more protein-nucleosome adducts or complexes.

Histone modifications are not equally distributed in chromatin and some modifications will therefore occur more frequently than others in a biological sample. Where a calibrant is being produced for a nucleosome moiety containing an uncommon histone modification, samples of biologically derived nucleosomes may contain a low, or a very low, proportion of the desired nucleosomes. Thus, the preparation of biologically derived calibration materials for nucleosomes containing some histone modifications, may require enrichment for those nucleosomes. Such enrichment may be effected by a number of means including; (i) isolation or purification of a nucleosome preparation for the desired nucleosomes; (ii) exposure of source nucleosome material to histone modifying enzymes (i.e. to produce nucleosomes with the desired modifications); (iii) modification (e.g. genetic modification or transient transfection) of cultured cells to over-express histone modifying enzymes (i.e. so that nucleosomes with the desired modifications are produced in greater quantities by the cultured cells); or (iv) treatment of cultured cells with a compound that modulates (i.e. increases or decreases) histone modifying enzyme activity or co-factors for enzymatic activity (v) any combination of these methods. These methods will be described in more detail below.

In one embodiment, a biologically derived nucleosome preparation enriched for nucleosomes containing a particular (low abundance) histone modification is prepared by isolation of, or purification for, the desired nucleosomes. For example, in a positive enrichment embodiment this is achieved by chromatin immunoprecipitation of nucleosomes containing the histone modification of interest. Methods for chromatin immunoprecipitation are well known in the art. In a typical method, an antibody that binds selectively to the histone modification of interest is immobilised on a solid phase support (for example, without limitation, agarose or sepharose or other particulate materials) and contacted with the source nucleosome material. Nucleosomes containing the histone modification of interest are isolated from other nucleosomes on the solid phase. The solid phase nucleosomes may be used in suspension as a calibrant (i.e. as the biologically derived nucleosome preparation). Alternatively, the purified nucleosomes may be removed from the solid phase for use as a liquid phase calibrant. Such an enrichment process may be applied to a nucleosome preparation derived from any natural source.

In a negative enrichment embodiment, a particular histone modification is enriched by removal or depletion of other nucleosomes from the preparation. In this embodiment an antibody that selectively binds to a nucleosome not containing the histone modification of interest is immobilised on a solid phase. The solid phase is contacted with the source nucleosome material and nucleosomes not containing the histone modification of interest are removed from the nucleosome preparation, thus enriching the preparation for nucleosomes containing the desired histone modification. For illustrative purposes only, an example of how this may be achieved is to select an antibody, or antibodies, that bind to other possible state(s) of the histone at the (same) amino acid position(s). For example, if the nucleosomes of interest were those containing histone H3 acetylated at lysine 9 (H3K9Ac), then antibodies binding to any or all of unmodified H3K9, any methylated (Me) H3K9 moiety (H3K9Me, H3K9Me2 or H3K9Me3) or any other post translationally modified (PTM) lysine moiety. Many such post translational modifications of lysine are known in the art. Such an enrichment process may be applied to a nucleosome preparation derived from any natural source.

In a further embodiment, a biologically derived nucleosome preparation enriched for nucleosomes containing a particular histone modification is prepared by enzymatic conversion of (other) nucleosomes into the nucleosomes of interest. This may be achieved using enzymes known in the art including, without limitation, histone methyl transferase (HMT), histone acetyl transferase (HAT), histone demethylase, histone deacetylase or other histone modifying enzymes. A large number of histone PTMs are known in the art and any enzyme effecting any PTM may be used for this purpose in methods of the invention. In this embodiment a histone modifying enzyme is added to a biologically derived nucleosome preparation which acts as a substrate for the enzyme. It will be understood that the histone modifying enzyme may also be applied directed to a cell culture from which the nucleosome preparation is subsequently derived. The presence of the enzyme alters the PTM of histones within the nucleosomes. Selection of the appropriate enzyme(s) used leads to conversion of nucleosome associated histone to the histone modification of interest. Using the same example (H3K9Ac) as above: a biologically derived nucleosome preparation may be enriched for nucleosomes containing this PTM using a HAT enzyme, optionally in combination with other enzymes (for example a histone demethylase enzyme). Such an enrichment process may be applied to a nucleosome preparation derived from any natural source.

Therefore, in one embodiment, the biologically derived nucleosomes are obtained from nuclease digests of cells grown in culture, said cells being immortalised human or animal cells or primary human or animal cells whereby the distribution of DNA modifications and/or histone variants and/or histone post translational modifications is adapted by treatment with one or more enzymes (e.g., Histone Acetyl Transferase, Histone Methyl Transferase or DNA methyl transferase).

In a further embodiment, cells in culture are transiently or stably genetically modified so that they over-express one or more histone modifying enzymes. The genetically modified cell line may be produced in any way known in the art, including by stably incorporating suitable gene sequences encoding histone modifying enzymes into the genome of the cell or by transfecting the cells with a suitable vector or plasmid containing such gene sequences. The advantage of this embodiment is that chromatin harvested from the cell line may be enriched for particular post-translational modifications. Many such histone modifying enzymes are known in the art and any histone modifying enzyme may be used for the methods described herein. Using the same example (H3K9Ac) as above: a cell may be engineered to produce chromatin containing elevated levels of nucleosomes containing H3K9Ac by stable or transient transfection of genetic sequences encoding a HAT enzyme, optionally in combination with genetic sequences for other enzymes (for example a histone demethylase enzyme).

In a further embodiment, a genetically modified animal is produced that over expresses one or more histone modifying enzymes. Chicken erythrocyte cells are a commonly used source of biologically derived nucleosomes. Thus, for example, a genetically modified chicken may be used to produce nucleosomes that contain high level(s) of particular histone modification(s).

In one embodiment, cells in culture are treated with a compound that modulates (i.e. increases or decreases) histone modifying enzyme activity or co-factors for enzymatic activity. Such compounds include, for example, histone methyltransferase and demethylase inhibitors.

In one embodiment, the biologically derived nucleosomes are obtained from an animal source where some or substantially all of the native DNA and associated modifications are removed and replaced with a synthetic DNA sequence. Said synthetic sequence may include, but is not limited to, a Widom nucleosome positioning sequence, a DNA sequence containing a disease specific single nucleotide polymorphism (SNP) (e.g. a KRAS). The length of the synthetic DNA sequence may be selected to provide nucleosomes with discrete DNA length e.g. 147 base pairs (bp), 167 bp or 187 bp of double stranded DNA. The synthetic DNA sequence can include a unique SNP or combination of SNPs to act as a barcode for identification. The synthetic DNA sequence can be blunt ended or selected to have a specific overhang whereby the overhang could represent a sequence specific to a physiological cleavage site enriched in a particular disease state e.g. cancer.

### Binding Agents

The method described herein use binding agents to perform the assays.

In one embodiment, the binding agent is a chromatin protein. In preferred embodiments, the binding agent is an antibody. In one embodiment, the assay employs a single binding agent. In another embodiment, the immunoassay is a 2-site immunometric (or sandwich) assay employing two binding agents, such as antibodies. The antibodies or other binding agents may be directed to bind to any epitope present in a nucleosome including without limitation binding to a histone, nucleosome core or DNA epitope. In some embodiments in which nucleosome adducts are measured, one or more antibodies may be directed to bind to a protein adducted to a nucleosome. In one embodiment, the binding agent is directed to a post-translational modification of a histone.

### Assays

In one embodiment, the assay is an immunoassay. The immunoassays described herein include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels. All of said immunoassay methods are well known in the art, see for example Salgame et al, 1997, supra and van Nieuwenhuijze et al, 2003, supra.

Identifying, detecting and/or quantifying can be performed by any method suitable to identify the presence and/or amount of a specific protein in a biological sample from a subject or a purification or extract of a biological sample or a dilution thereof. In particular, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner. The present invention finds particular use in plasma samples which may be obtained from the subject.

Identification, detection and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed by one or more method(s) selected from the group consisting of: SELDI (-TOF), MALDI (-TOF), a 1-D gel-based analysis, a 2-D gel-based analysis, Mass spec (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

According to another aspect of the invention, there is provided a method for the measurement of the level of nucleosomes or histone associated with nucleosomes in a biological sample, the method comprising:
(i) obtaining a sample of a biological fluid;
(ii) contacting the sample with a binding agent to the nucleosomes;
(iii) measuring a parameter or degree of nucleosome binding of the binding agent to the nucleosome using an instrument adapted for conducting the assay;
(iv) comparing the parameter or degree of nucleosome binding obtained in step (iii) with standardisation data; and
(v) using the comparison in step (iv) to convert the parameter or degree of nucleosome binding measured in step (iii) into an amount or concentration of nucleosome present in the sample.

In one embodiment, the standardisation data is stored in the instrument or retrieved from a remote location (i.e. a location separate to the instrument). Preferably, the standardisation data is retrieved electronically from the remote location. For example, the standardisation data (which may also be referred to as the calibration data) may be provided on a storage device. Such storage devices include, but are not limited to, a USB flash drive, hard-drive, memory card, DVD or CD-ROM. Alternatively, the standardisation data may be downloaded from a remote location via a communication network (for example, the internet).

The method may be used to measure the nucleosome and components thereof, as described herein. In one embodiment, the method is for the measurement of the level of a histone post translational modification in a sample.

References to “subject” or “patient” are used interchangeably herein. The subject may be a human or an animal subject. In one embodiment, the subject is a human. In one embodiment, the subject is a (non-human) animal. In one embodiment, the subject is a non-human mammal, such as a dog, mouse, rat or horse, in particular a dog. The methods described herein may be performed in vitro, in vivo or ex vivo.

### Additional Biomarkers

The level of cell free nucleosomes may be detected or measured as one of a panel of measurements. The panel may comprise different epigenetic features of the nucleosome as described hereinbefore (e.g. a histone isoform and a PTM). In one embodiment, the panel comprises one or more cytokines, such as one or more interleukins.

### Diagnostic Methods

The identification of biomarkers for a disease state permits integration of diagnostic procedures and therapeutic regimes. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker using a method of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

In one embodiment, the method is a clinical diagnostic test for a human or animal subject.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

The method may be used to monitor a patient. Therefore, in one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission. Therefore, in one embodiment the method is repeated on one or more occasions and any changes in the level of cell free nucleosomes or component thereof is used to monitor the progression of a disease in the patient.

### Kits

Kits (or panels) are provided for performing methods of the invention. Such kits will suitably comprise one or more ligands for detection and/or quantification of a target biomarker, optionally together with instructions for use of the kit.

According to a further embodiment, there is provided a kit for quantifying the amount of a nucleosome or a histone associated with a nucleosome in a biological sample wherein the kit comprises an automated immunoassay analyser, a nucleosome binder and electronically accessible standardisation data and does not comprise a nucleosome standard or calibrant.

According to a further embodiment, there is provided a kit for measuring the level of nucleosomes or histone associated with nucleosomes in a biological sample, wherein the kit comprises an automated immunoassay analyser, a nucleosome binder and electronically accessible standardisation data and does not comprise a nucleosome standard or calibrant.

### EXAMPLES

### Example 1: Onboard Calibration of Nu.Q H3.1 Chemiluminescence Assay

Five contrived plasma samples were generated from human K2EDTA plasma, previously confirmed to contain low levels of H3.1 variant nucleosomes, spiked with increasing concentrations of HeLa mononucleosomes and designated iQC1-5, were prepared as reference samples. The samples were aliquoted and frozen at −80° C. until the day of testing. Two kit controls, comprising H3.1 recombinant nucleosomes, freeze dried from TEA (Tri-ethylamine) buffer (TEA 10 mM pH7.5, NaCl 20 mM, EDTA 0.1 mM, 1% BSA) were designated KC1 and KC2.

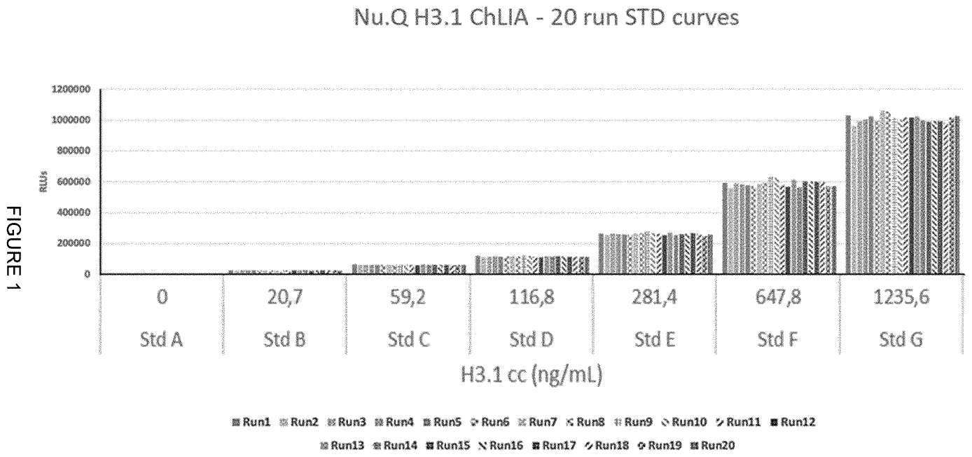

A 7 point standard curve was prepared by rehydrating known quantities of freeze dried H3.1 recombinant nucleosomes from TEA buffer in deionised water. The standard curve was analysed twice daily for 10 days on an DS i10 next generation immunoanalyzer (Immunodiagnostic Systems) and an overlay of the 20 curves is shown in FIG. 1. The intra-run CVs between duplicates on the standard curve was 3.2% or less whilst the inter run CVs were below 5.3% (Table 1).

### Assay Details

Samples iQC1-5, KC1 and KC2 were analysed twice daily in duplicate over the same 10-day period (40 runs per sample in total) and quantified using either the standard curve generated immediately before samples were run, or the first standard curve from day 1. The highest intra run CV (between duplicates) for the standard curves was 3.2% and the highest inter run cv was 5.3% (Table 1). A comparison of the concentrations quantified using the daily calibration curve compared to the single (day 1) pre-calibration curve is shown in Table 2. The intra run CVs between duplicates was 2.6% or less with both single (on board) pre-calibration and daily calibration and less than 4.7% between runs using the single pre-calibration. With daily calibration the highest between run CV was 5% demonstrating that a single (on board) pre-calibration curve generated at day 1 allowed accurate quantification for H3.1 nucleosomes in samples for at least 10 days with a concentration bias of 2% or less between the single calibration compared to daily calibration (Table 2).

TABLE 1
Intra-run (between duplicate) and inter-run CVs for
each point of a 7 point standard curve comprising known
quantities of recombinant H3.1 nucleosomes in TEA buffer
and quantified on an IDA-i10 Chemiluminescence analyser.

| | | Mean | Intra-run | Inter-run |
|---|---|---|---|---|
| STD | n | RLU | CV | CV |
| STD A | 40 | 3189 | 3.2% | 5.3% |
| STD B | 40 | 24312 | 2.0% | 2.7% |
| STD C | 40 | 62071 | 1.3% | 2.1% |
| STD D | 40 | 115695 | 1.6% | 2.7% |
| STD E | 40 | 261466 | 1.0% | 2.7% |
| STD F | 40 | 588884 | 1.5% | 3.6% |
| STD G | 40 | 1009956 | 1.5% | 2.6% |

TABLE 2
Quantification of IQC1-5 reference samples (native plasma samples spiked or non-spiked with HeLa nucleosomes)
and kit controls KC1 and KC2 (recombinant H3.1 nucleosomes spiked into nucleosome negative plasma) using
separate pre-run calibration curves (Daily Calibration) or pre-calibration (day 1) standard curve (Single
Calibration). Intra run (between duplicate) and inter run CVs are shown for Daily and Single calibration
together with the apparent concentration bias between the two calibration methods.

| | | Concentration Precision | | | | Concentration Precision | | | | |
|---|---|---|---|---|---|---|---|---|---|---|
| | | (Daily calibration) | | | | (Single Calibration) | | | | |
| | | | Mean cc | Intra- | Inter- | | Mean cc | Intra- | Inter- | Concentration |
| | n | SAMPLE | (ng/mL) | Run CV | Run CV | SAMPLE | (ng/mL) | Run CV | Run CV | bias |
| iQCs | 40 | IQC1 | 14.5 | 2.0% | 5.0% | IQC1 | 427.2 | 2.6% | 3.7% | 2.0% |
| | 40 | IQC2 | 50.9 | 1.9% | 3.4% | IQC2 | 14.4 | 2.0% | 4.7% | 1.0% |
| | 40 | IQC3 | 203.5 | 2.3% | 4.0% | IQC3 | 50.2 | 1.9% | 3.3% | 1.4% |
| | 40 | IQC4 | 435.9 | 2.6% | 3.9% | IQC4 | 199.7 | 2.3% | 4.0% | 1.9% |
| | 40 | IQC5 | 723.1 | 2.0% | 3.5% | IQC5 | 709.6 | 2.1% | 3.3% | 1.9% |
| KC | 40 | KC 1 | 50.6 | 1.7% | 2.4% | KC 1 | 49.9 | 1.7% | 3.2% | 1.3% |
| | 40 | KC2 | 325.4 | 1.7% | 2.9% | KC2 | 318.9 | 1.6% | 2.7% | 2.0% |
| | | | | | | | | | | 1.7% |

### Example 2: Onboard Calibration of Nu.Q H3K36Me3 Chemiluminescence Assay

Four contrived plasma samples were generated from human K2EDTA plasma, previously confirmed to contain low levels of H3K36Me3 modified nucleosomes (<10 ngmL-1, IQC1) spiked with increasing concentrations of HeLa mononucleosomes (IQC2 and IQC4) or recombinant H3K36Me3 nucleosomes (IQC5), were prepared as reference samples. The samples were aliquoted and frozen at −80° C. until the day of testing. Two kit controls, comprising recombinant H3K36Me3 nucleosomes, freeze dried from TEA buffer+5% Trehalose (TEA 10 mM pH7.5, NaCl 20 mM, EDTA 0.1 mM, 1% BSA, 5% Trehalose) were designated KC1 and KC2.

A 7 point standard curve was prepared by rehydrating freeze dried recombinant H3K36Me3 nucleosomes from TEA buffer+5% Trehalose in deionised water. The standard curve was analysed once daily for 10 days on an iDS i10 next generation immunoanalyzer (supplier) to generate 10 separate standard curves.

### Assay Details

Samples iQC1-5, KC1 and KC2 were analysed twice daily in triplicate over the same 10-day (60 runs per sample in total) period and quantified using either the standard curve generated on the same day the samples were run or the first standard curve from day 1. A comparison of the concentrations quantified using the daily calibration curve compared to the single (day 1) pre-calibration curve is shown in Table 3. The intra run CVs between duplicates was 3.7% or less with both single (on board) pre-calibration and daily calibration and less than 7.6% between runs demonstrating that a single (on board) pre-calibration curve generated at day 1 allowed accurate quantification for H3K36Me3 nucleosomes in samples for at least 10 days with an average concentration bias of less than 5.2% between the single calibration compared to daily calibration. (Table 3).

TABLE 3
Quantification of IQC1-5 reference samples (native plasma samples spiked or non-spiked with HeLa nucleosomes)
and kit controls KC1 and KC2 (recombinant H3K36Me3 nucleosomes spiked into nucleosome negative plasma)
using separate daily calibration curves (Daily Calibration) or pre-calibration (day 1) standard curve
(Single Calibration). Intra run (between duplicate) and inter run CVs are shown for Daily and Single
calibration together with the apparent concentration bias between the two calibration methods.

| | | Concentration Precision | | | | Concentration Precision | | | | |
|---|---|---|---|---|---|---|---|---|---|---|
| | | (Daily calibration) | | | | (Single Calibration) | | | | |
| | | | Mean cc | Intra- | Inter- | | Mean cc | Intra- | Inter- | Concentration |
| | n | SAMPLE | (ng/mL) | Run CV | Run CV | SAMPLE | (ng/mL) | Run CV | Run CV | bias |
| iQCs | 60 | IQC1 | 8.3 | 2.8% | 7.0% | IQC1 | 8.1 | 2.7% | 6.4% | 2.5% |
| | 60 | IQC2 | 134.8 | 1.9% | 4.6% | IQC2 | 127.3 | 1.9% | 5.4% | 5.9% |
| | 60 | IQC4 | 259.5 | 3.4% | 5.8% | IQC4 | 244.5 | 3.7% | 7.6% | 6.1% |
| | 60 | IQC5 | 665.0 | 2.0% | 5.3% | IQC5 | 628.0 | 2.0% | 5.9% | 5.9% |
| KC | 60 | KC 1 | 43.4 | 3.3% | 6.3% | KC 1 | 41.5 | 3.2% | 6.1% | 4.6% |
| | 60 | KC2 | 289.5 | 1.9% | 4.2% | KC2 | 272.6 | 1.9% | 5.1% | 6.2% |
| | | | | | | | | | | 5.2% |

### Example 3: Batch Calibration of Automated Immunoassay Cartridges

Five contrived plasma samples were generated from human K2EDTA plasma, previously confirmed to contain low levels of H3K36me3 modified nucleosomes, spiked with increasing concentrations of recombinant H3K36Me3 nucleosomes and designated iQC1-5, were prepared as reference samples. The samples were aliquoted and frozen at −80° C. until the day of testing. Two kit controls, comprising H3K36Me3 nucleosomes, freeze dried from TEA buffer+5% Trehalose (TEA 10 mM pH7.5, NaCl 20 mM, EDTA 0.1 mM, 1% BSA, 5% Trehalose) were designated KC1 and KC2.

A 7 point standard curve was prepared by rehydrating the freeze dried H3K36Me3 nucleosomes from TEA buffer+5% Trehalose (TEA 10 mM pH7.5, NaCl 20 mM, EDTA 0.1 mM, 1% BSA, 5% Trehalose in deionised water. A single batch of 500 Proteinsimple Simple Plex cartridges were prepared by coating anti-H3K36Me3 capture antibody on Glass Nano Reactors which were then placed, sequentially in triplicate, within the microfluidic channels of Singleplex cartridges configured to run up to 32 samples. Fluorescently labelled Anti-nucleosome detection antibody, which recognises a conformational epitope on intact nucleosomes, was preloaded onto the cartridge.

The standard curve was analysed on a randomly selected subset of 50 cartridges on a semi-automated immunoanalyzer (Proteinsimple Ella, Biotechne) and the data combined to generate an average standard curve—designated as the factory calibrated standard curve. The average variability between standard curves run on cartridges was less than 10%.

Samples iQC1-5, KC1 and KC2 were analysed twice daily over the same 10-day period in two wells together with the 7 standards to generate an on-board standard curve. The samples and KC1 and KC2 were quantified using either the on-board standard curve generated on the day the samples were run or the factory calibrated standard curve. The between well CVs was less than 10% with both on board and factory calibrated standard and less than 20% between runs demonstrating that the factory calibrated standard curve generated allowed accurate quantification for H3K36Me3 nucleosomes in samples with an average concentration bias of less than 10% between the on-board standard and factory calibrated standard.

### Example 4: Factory Calibration of Point of Care Devices

Five contrived plasma samples were generated from human K2EDTA plasma, previously confirmed to contain low levels of H3.1 variant nucleosomes, spiked with increasing concentrations of HeLa mononucleosomes and designated iQC1-5. were prepared as reference samples. The samples were aliquoted and frozen at −80° C. until the day of testing. Two kit controls, comprising H3.1 nucleosomes, freeze dried from TEA (Tri-ethylamine) buffer (TEA 10 mM pH7.5, NaCl 20 mM, EDTA 0.1 mM, 1% BSA) were designated KC1 and KC2.

A 7 point standard curve was prepared by rehydrating the freeze dried H3.1 nucleosomes from TEA (Tri-ethylamine) buffer (TEA 10 mM pH7.5, NaCl 20 mM, EDTA 0.1 mM, 1% BSA) in deionised water. A single batch of 300 LightDeck® cartridges were prepared by spotting anti-H3.1 capture antibody onto planar wave guides in duplicate. Fluorescently labelled anti-nucleosome detection antibody, which recognises a conformational epitope on intact nucleosomes, was freeze dried and preloaded into the cartridge sample entry port.

The standard curve was analysed on a randomly selected subset of 40 cartridges on a point of care analyzer (LightDeck) and the data combined to generate an average standard curve—designated as the factory calibrated standard curve. The average variability between standard curves run on cartridges was less than 9%.

Samples iQC1-5, KC1 and KC2 were analysed twice daily over a 10-day period on single cartridges. The samples and KC1 and KC2 were quantified using the factory calibrated standard curve. The inter run sample CVs was less than 15% demonstrating that the factory calibrated standard curve allowed accurate quantification for H3.1 nucleosomes.

### Example 5: Factory Calibration of Point of Care Devices

Five contrived plasma samples were generated from human K2EDTA plasma, previously confirmed to contain low levels of H3R8 citrulline modified nucleosomes, spiked with increasing concentrations of H3K8Cit recombinant nucleosomes and designated iQC1-5, were prepared as reference samples. The samples were aliquoted and frozen at −80° C. until the day of testing. Two kit controls, comprising recombinant H3R8 citrulline modified nucleosomes, freeze dried from TEA (Tri-ethylamine) buffer+5% Trehalose (TEA 10 mM pH7.5, NaCl 20 mM, EDTA 0.1 mM, 1% BSA, 5% Trehalose) were designated KC1 and KC2.

A 7 point standard curve was prepared by rehydrating freeze dried recombinant H3R8 citrulline modified nucleosomes from TEA (Tri-ethylamine) buffer+5% Trehalose (TEA 10 mM pH7.5, NaCl 20 mM, EDTA 0.1 mM, 1% BSA, 5% Trehalose) in deionised. A single batch of 300 LightDeck® cartridges were prepared by spotting anti-H3R8 citrulline capture antibody onto planar wave guides in duplicate. Fluorescently labelled anti-nucleosome detection antibody, which recognises a conformational epitope on intact nucleosomes, was freeze dried and preloaded into the cartridge sample entry port.

The standard curve was analysed on a randomly selected subset of 40 cartridges on a point of care analyzer (LightDeck) and the data combined to generate an average standard curve—designated as the factory calibrated standard curve. The average variability between standard curves run on cartridges was less than 9%.

Samples iQC1-5, KC1 and KC2 were analysed twice daily over a 10-day period on single cartridges. The samples and KC1 and KC2 were quantified using the factory calibrated standard curve. The inter run sample CVs was less than 15% demonstrating that the factory calibrated standard curve allowed accurate quantification for H3R8 citrulline containing nucleosomes.

1. A method for quantifying an amount of a nucleosome or a histone associated with a nucleosome in a biological sample, the method comprising:
(i) providing a sample of nucleosomes at various concentrations to create a reference standard wherein the nucleosomes are assayed to generate a standard curve in a pre-calibration step;
(ii) adding a binding agent to a biological sample to perform an assay to measure an amount of nucleosome or histone associated with the nucleosome in the biological sample; and
(iii) quantifying the amount of nucleosome or histone associated with the nucleosome in the biological sample by measuring the amount of binding agent bound in the biological sample and comparing the amount to the reference standard.

2. The method according to claim 1, wherein the sample of nucleosomes used in the precalibration step are biologically derived or recombinant.

3. The method according to claim 1, wherein the pre-calibration is factory calibration.

4. The method according to claim 1, wherein the pre-calibration step is carried out at least 10 days before step (ii).

5. A method for measuring a level of nucleosomes or histone associated with nucleosomes in a biological sample, the method comprising:
(i) obtaining a sample of a biological fluid;
(ii) contacting the sample with a binding agent to the nucleosomes;
(iii) measuring a parameter or degree of nucleosome binding of the binding agent to the nucleosome using an instrument;
(iv) comparing the parameter or degree of nucleosome binding obtained in step (iii) with standardisation data; and
(v) using the comparison in step (iv) to convert the parameter or degree of nucleosome binding measured in step (iii) into an amount or concentration of nucleosome present in the sample.

6. The method according to claim 5, wherein the standardisation data is retrieved electronically from a remote location.

7. The method according to claim 1, wherein the assay is an immunoassay.

8. The method according to claim 7, wherein the assay employs a single binding agent.

9. The method according to claim 7, wherein the assay is a 2-site immunometric assay employing two binding agents.

10. The method according to claim 1, wherein the binding agent binds a histone, a nucleosome core, a DNA epitope, a post-translational modification of a histone, or a protein adducted to a nucleosome.

11-13. (canceled)

14. The method according to claim 2, wherein the biologically derived or recombinant nucleosome contains a nucleosomes comprising a specified epigenetic feature or a multiplicity of nucleosome types each comprising one or more specified epigenetic feature.

15-16. (canceled)

17. The method according to claim 14, wherein the one or more specified epigenetic feature is a histone post translational modification, a histone variant, a nucleotide or modified nucleotide or a protein-nucleosome adduct.

18. (canceled)

19. The method according to claim 17, wherein the histone post translational modification is selected from the group consisting of acetylation, methylation or ubiquitination of lysine residues; citrullination or methylation of arginine residues; and phosphorylation of serine residues.

20-25. (canceled)

26. The method according to claim 1, wherein the nucleosome reference sample comprises a recombinant nucleosome.

27. The method according to claim 26, wherein the recombinant nucleosome is a semisynthetic nucleosome or wherein at least part of the recombinant nucleosome is synthesized.

28-29. (canceled)

30. The method according to claim 1, wherein the nucleosome reference sample is a biologically derived nucleosome reference sample.

31. (canceled)

32. The method according to claim 30, wherein the biologically derived nucleosome preparation is prepared by (i) isolation or purification of a nucleosome preparation for the desired nucleosomes; (ii) exposure of source nucleosome material to histone modifying enzymes; (iii) modification of cultured cells to over-express histone modifying enzymes; and/or (iv) treatment of cultured cells with a compound that modulates histone modifying enzyme activity.

33. The method according to claim 1, wherein the method is a clinical diagnostic test for a human or animal subject.

34. The method according to claim 33, wherein the biological sample is a human or animal body fluid sample.

35. (canceled)

36. A kit for measuring the level of nucleosomes or histone associated with nucleosomes in a biological sample or for quantifying an amount of a nucleosome or a histone associated with a nucleosome in a biological sample, wherein the kit comprises an automated immunoassay analyser, a nucleosome binder and electronically accessible standardisation data and does not comprise a nucleosome standard or calibrant.
